(19) United States
(12) Patent Application Publication
(10) Pub. No.: US 2003/0045853 A1
(43) Pub. Date: Mar. 6, 2003

(54) Pocket design for absorbent article

(76) Inventor: Barbara O. Sauer, Fremont, WI (US)

Correspondence Address: Mark E. Fejer, Pauley Petersen Kinne & Fejer, Suite 365, 2800 W. Higgins Road, Hoffman Estates, IL, 60195, US

(21) Appl. No.: 09/745,006

(22) Filed: Dec. 20, 2000

Related U.S. Application Data
(60) Provisional application No. 60/173,026, filed on Dec. 23, 1999.

(51) Int. Cl. A61F 13/15

(52) U.S. Cl. 604/385.19; 604/385.06

(57) ABSTRACT

An absorbent article having a fluid impermeable backsheet, a body-side liner attached to the fluid impermeable backsheet and a flap sheet attached to at least one of the body-side liner and the fluid impermeable backsheet and forming a pocket. The flap sheet includes at least two longitudinally oriented discrete pleats disposed proximate a back region of the absorbent article on opposed sides of the pocket.

### FIELD OF THE INVENTION

This invention relates to diapers or other absorbent articles, such as training pants, incontinence garments and the like and, more particularly, to a diaper or absorbent article having means for isolation and containment of waste material from the body.

### BACKGROUND OF THE INVENTION

A typical disposable diaper or other absorbent article comprises a fluid-permeable top sheet, a fluid-impermeable backsheet, an absorbent assembly between the top sheet and the backsheet, and a means for fastening the diaper or article to the wearer. Although current diapers or other absorbent articles have been generally accepted by the public, there is still a need for improvement in certain areas, particularly in the area of isolating and containing waste material away from the skin of the wearer. Materials used in the manufacture of such disposable articles include polymeric films and nonwovens, including spunbond fibers or webs, meltblown fibers or webs and bonded carded webs. Although current diapers or absorbent articles have been generally satisfactory in preventing leakage of urine outside of the diaper or absorbent article, the waste material contained therein too often is in contact with the skin of the wearer. If this contact between waste material and the skin is prolonged over a period of time, it can cause undesirable effects such as skin dermatitis, skin hydration, or messy clean-ups.

One solution to this problem is the integration of a pocket into the diaper or absorbent article for collection of solid fecal material. Such a pocket, disposed toward the back of the diaper or absorbent article prevents leaks, especially of feces. The pocket is formed by a multi-layer material comprising a fluid permeable top sheet, a fluid impermeable backsheet and an absorbent layer disposed between the top sheet and the backsheet. Extra material in the top sheet and the backsheet create a pocket or depression for feces to collect in away from the skin of the wearer. U.S. Pat. No. 5,462,541 teaches a pocket-like diaper or absorbent article comprising a backsheet, a top sheet having an opening, an absorbent between the backsheet and the top sheet, and an elevating device below the top sheet that spaces the top sheet above the absorbent, thereby forming a pocket-like shape for isolating and containing waste material.

For diapers and absorbent articles having pockets, it is preferred to have the width of the pocket transverse nearly the entire width of the back of the product. This long width has at least three key benefits. First, it has the potential for tying the back flap into the fastener system. Second, it maximizes the range of motion (or how wide the pocket can open) at the center of the pocket or flap, since the center is far away from the bonded side edges. Third, it protects against leakage across the entire back width. The disadvantage of a long width design is that more material is required, thereby resulting in higher cost than a narrower pocket.

Having extra material in the top sheet and backsheet to provide room for containment of solids the entire length of the product has several drawbacks including extra material costs and material bunching where a solids void volume is not needed, such as the front of the product where liquid exudates are excreted.

### SUMMARY OF THE INVENTION

It is one object of this invention to provide a disposable absorbent article, such as a disposable diaper, training pants, incontinence garments and the like which includes a pocket for isolation of solid wastes from the body of the wearer which addresses the issues discussed above.

It is one object of this invention to provide a method for producing a diaper or absorbent article having a pocket, which method utilizes no more material than the amount of material required for producing a diaper or other absorbent article having a conventional pocket design which extends the full length of the article.

These and other objects are addressed by an absorbent article comprising a fluid impermeable outer cover or backsheet, a fluid permeable body-side liner attached to a face (also referred to as side) of the outer cover, and pocket means for forming a depression or pocket for collection of the fecal matter. The pocket means comprises a flap sheet or pocket sheet attached to the body-side liner, whereby the body-side liner is disposed between the fluid impermeable outer cover and the flap sheet. To provide sufficient transverse dimension to the pocket or to enlarge the volume of the pocket, discrete pleats are incorporated into the flap sheet along longitudinal sides of the pocket, which pleats are disposed proximate a rear waist section of said absorbent article. In accordance with one embodiment of this invention, the body-side liner is a multi-layer material comprising a fluid permeable top sheet and an absorbent layer, said absorbent layer being disposed between the fluid impermeable outer cover and the fluid permeable top sheet.

A method for producing a diaper or other absorbent article having a pocket for isolation and containment of solid fecal matter away from the body of the wearer in accordance with one embodiment of this invention comprises forming a substantially rectangular multi-layer material comprising a fluid impermeable backsheet and a body-side liner and removing a leg cut out portion from opposed longitudinal edges of the material resulting in formation of two enlarged end portions and a narrowed intermediate portion. A flap sheet or pocket sheet is attached to the body-side liner proximate an end of the substantially rectangular multi-layer material corresponding to a rear waist section of the absorbent article and longitudinally pleated on both sides of a longitudinal centerline of the multi-layer material, thereby enabling expansion of the flap sheet away from the fluid impermeable backsheet and the skin of a wearer as necessary for the containment of fecal matter.

### BRIEF DESCRIPTION OF THE DRAWINGS

These and other objects and features of this invention will be better understood from the following detailed description taken in conjunction with the drawings wherein:

FIG. 1 is a top plan view of one embodiment of this invention;

FIG. 2 is a schematic diagram of a cross-sectional view from the perspective of line II-II in FIG. 1 showing formation of a pocket with pleated sides in accordance with one embodiment of this invention; and

FIG. 3 is a cross-sectional view corresponding to FIG. 2 showing a pocket with pleated sides in accordance with another embodiment of this invention.

### DETAILED DESCRIPTION OF PRESENTLY PREFERRED EMBODIMENTS

Definitions

As used herein, the term “comprising” is inclusive or open-ended and does not exclude additional unrecited elements, compositional components, or method steps.

As used herein, the term “nonwoven” or “nonwoven web” means a structure of individual fibers or threads which are interlaid, but not in an identifiable repeating manner. Nonwoven webs have been, in the past, formed by a variety of processes such as, for example, meltblowing processes, spunbonding processes, coforming processes, hydroentangling, air-laid and bonded carded web processes.

As used herein, the term “spunbond fibers” refers to small diameter fibers which are formed by extruding molten thermoplastic material as filaments from a plurality of fine, usually circular capillaries of a spinneret, with the diameter of the extruded filaments then being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992 and 3,341,394 to Kinney, U.S. Pat. No. 3,502,763 to Hartmann, and U.S. Pat. No. 3,542,615 to Dobo et al. Spunbond fibers are generally not tacky when they are deposited onto a collecting surface. Spunbond fibers are generally continuous and have average diameters (from a sample of at least 10 fibers) larger than 7 microns, more particularly, between about 10 and 30 microns. The fibers may also have shapes such as those described in U.S. Pat. No. 5,277,976 to Hogle et al., U.S. Pat. No. 5,466,410 to Hills, and U.S. Pat. No. 5,069,970 and U.S. Pat. No. 5,057,368 to Largman et al., which describe hybrids with unconventional shapes. A nonwoven web of spunbond fibers produced by melt spinning is referred to as a “spunbond”.

As used herein, the term “meltblown fibers” means fibers formed by extruding a molten thermoplastic material through a plurality of fine, usually circular, die capillaries as molten threads or filaments into converging high velocity, usually hot, gas (for example, air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, by U.S. Pat. No. 3,849,241 to Butin et al. Meltblown fibers are microfibers which may be continuous or discontinuous, are generally smaller than 10 microns in average diameter.

As used herein, the term “bonded carded web” refers to webs made from staple fibers which are sent through a combing or carding unit, which breaks apart and aligns the staple fibers in the machine direction to form a generally machine direction-oriented fibrous nonwoven web. Such fibers are usually purchased in bales which are placed in a picker or fiberizer which separates the fibers prior to the carding unit. Once the web is formed, it is then bonded by one or more of several known bonding methods.

As used herein, the term “polymer” generally includes, but is not limited to, homopolymers, copolymers, such as for example, block, graft, random and alternating copolymers, terpolymers, etc., and blends and modifications thereof. In addition, unless otherwise specifically limited, the term “polymer” also includes all possible geometric configurations of the molecule. These configurations include, but are not limited to, isotactic, atactic, syndiotactic and random symmetries.

As used herein, the term “microfibers” refers to small diameter fibers having an average diameter not greater than about 100 microns, for example, having an average diameter of from about 0.5 microns to about 50 microns, or more particularly, having an average diameter of from about 2 microns to about 40 microns. Another frequently used expression of fiber diameter is denier, which is defined as grams per 9000 meters of a fiber, and may be calculated as fiber diameter in microns squared, multiplied by the density in grams/cc, multiplied by 0.00707. A lower denier indicates a finer fiber and a higher denier indicates a thicker or heavier fiber. For example, a diameter of a polypropylene fiber given as 15 microns may be converted to denier by squaring, multiplying the results by 0.89 g/cc and multiplying by 0.00707. Thus, a 15 micron polypropylene fiber has a denier of about 1.42. Outside the United States, the unit of measurement is more commonly the “tex”, which is defined as the grams per kilometer of fiber. Tex may be calculated as denier/9.

The absorbent article of the present invention can be utilized as a baby diaper, adult incontinence garment, and the like. For purposes of the present discussion, the absorbent article will be described in terms of a baby diaper, but it is to be understood that the features and principles of the present invention may also be applied to other types of absorbent articles.

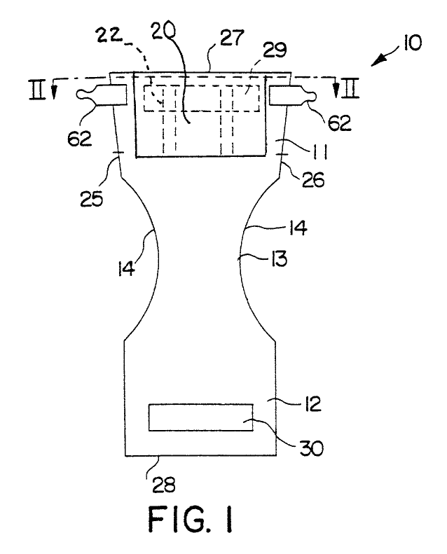

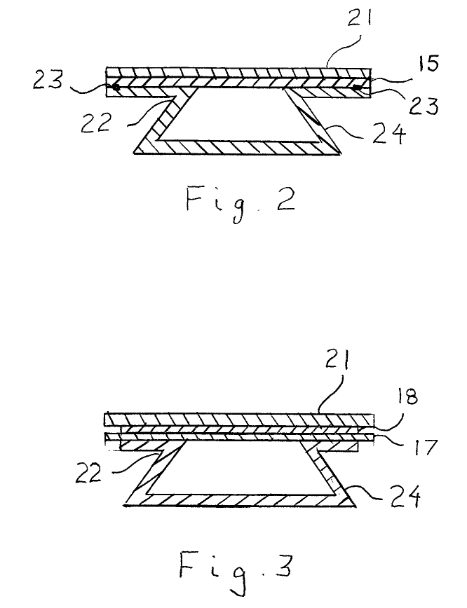

The invention disclosed herein in accordance with one embodiment is a disposable diaper comprising a pleated back pocket for containment of solid fecal matter away from the body of the wearer. Referring to FIG. 1, diaper 10 in accordance with one embodiment of this invention, shown in a flat, uncontracted state, comprises a material formed as a single piece having two enlarged end portions 11, 12 and a narrowed portion 13 intermediate the enlarged end portions 11, 12. For purposes of this description, enlarged end portion 12 corresponds to the front waist section of the diaper and enlarged end portion 11 corresponds to the back or rear waist section of the diaper. The front and rear waist sections include the general portions of the diaper which are constructed to extend substantially over the wearer's front and rear abdominal regions, respectively, during use. The narrowed portion of the diaper includes the general portion of the article which is constructed to extend through the wearer's crotch region between the legs. The opposed longitudinal side edges 25, 26 define leg cut outs 14 which preferably are curved or contoured to more closely fit the legs of the wearer. The opposed end edges 27, 28 define a waist opening for the diaper and typically are straight, although they may be curvilinear. The material further comprises a fluid impermeable backsheet or outer cover 21, a fluid permeable body-side liner attached to outer cover 21 and a flap sheet or pocket sheet 24 attached to the body-side liner 15 as shown in FIG. 2. In accordance with one embodiment of this invention, body-side liner 15 is a multi-layer material comprising a fluid permeable top sheet 17 and an absorbent layer 18 disposed between outer cover 21 and fluid permeable top sheet 17 as shown in FIG. 3. Marginal sections of backsheet 21 may extend past the terminal edges of the absorbent layer 16. The body-side liner 15 is generally coterminous with the backsheet 21 but may optionally cover an area which is smaller than backsheet 21. Flap sheet 24 may also be either coterminous with backsheet 21 or smaller than backsheet 21, as shown in FIG. 1. Flap sheet 24 is preferably fluid impermeable to prevent fluids which may be present in the body-side liner and feces which may be contained in the pocket from contacting the skin of the wearer. To provide better fit and to aid in reducing leakage, the longitudinal side edges 25, 26 and the end edges 27, 28 may be elasticized with suitable elastic members such as leg elastic members and waist elastic members 29, 30. For example, the leg elastic members may include single or multiple strands of elastic or elastomeric composites which are constructed to operably gather and shirr the longitudinal side edges 25, 26 of the diaper to provide elasticized leg bands which can fit closely around the legs of the wearer. Similarly, the waist elastic members 29, 30 can be employed to elasticize the end edges 27, 28 thereby providing elasticized waistbands.

The elastic members are secured to the material in an elastically contracted condition whereby in a normal under strain configuration, the elastic members effectively contract against the material.

The diaper as shown in FIG. 1 may further include a pair of fasteners or ears 62 which are used to secure the diaper around the waist of the wearer. Suitable fasteners include hook-and-loop type fasteners, adhesive type fasteners, buttons, pins, snaps and the like.

Known pocket designs for diapers having a narrow transverse dimension are generally unable to “open widely” to bridge the gap between the wearer's body and the body-side surface of the diaper. To address this problem, the pockets 20 of the absorbent articles of this invention are provided with discrete pleats 22 formed along opposed sides of flap sheet 24 as shown in FIG. 2 and are connected to the body-side liner 15, designated as reference numeral 23, by point bonding. By the term “discrete pleats,” we mean pleats formed by well-defined folds. It will, however, be apparent that other forms of bonding may be equally suitable. Thus, even though the transverse dimensions of the pocket may be somewhat narrower than traditional pockets, the material resulting from the pleating nevertheless permits the pocket to expand.

To produce a disposable absorbent article having a pocket in accordance with one embodiment of this invention, a substantially rectangular multi-layer material comprising a body-side liner and a fluid impermeable backsheet is formed and a portion of the material from opposed longitudinal edges is removed, forming two enlarged end portions and a narrowed intermediate portion. The narrowed intermediate portion forms leg cut outs along opposed edges of the narrowed intermediate portion. A flap sheet is attached to the body-side liner whereby a pocket is formed proximate one enlarged end portion corresponding to a back section of the disposable article. Discrete pleats are formed in the flap sheet on opposed sides of the pocket so as to extend in a direction parallel to a longitudinal centerline of the substantially rectangular multi-layer material.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

I claim:

1. An absorbent article comprising:
an elongated material having two enlarged end portions and a narrowed portion intermediate said two enlarged end portions, one of said two enlarged end portions corresponding to a back region of said absorbent article; and
said elongated material comprising a fluid impermeable backsheet, a fluid permeable body-side liner attached to one side of said fluid impermeable backsheet, and a flap sheet attached to said fluid permeable body-side liner, said flap sheet being expandable in a direction away from said fluid impermeable backsheet proximate said back region of said absorbent article, whereby a pocket for collection of fecal matter is formed.

2. An article in accordance with claim 1, wherein a width of said pocket traverses substantially an entire width of said back region.

3. An absorbent article in accordance with claim 1, wherein said flap sheet forms two opposed pleats along opposite sides of said pocket and disposed substantially parallel to a longitudinal centerline of said elongated material.

4. An absorbent article in accordance with claim 1, wherein at least one of said flap sheet, said fluid impermeable backsheet and said fluid permeable body-side liner comprises a nonwoven material.

5. An absorbent article in accordance with claim 4, wherein said nonwoven material is selected from the group consisting of spunbond, meltblown, bonded carded web and combinations thereof.

6. An absorbent article in accordance with claim 1, wherein said fluid permeable body-side liner is a multi-layer material comprising an absorbent layer and a fluid permeable top sheet, said absorbent layer being disposed between said fluid impermeable backsheet and said fluid permeable top sheet.

7. An absorbent article in accordance with claim 1, wherein said flap sheet is fluid impermeable.

8. A disposable diaper comprising:
a fluid impermeable outer cover, a fluid permeable body-side liner attached to said fluid impermeable outer cover and a flap sheet attached to said fluid permeable body-side liner, said flap sheet expandable in a direction away from said fluid impermeable outer cover whereby a pocket is formed inside said disposable diaper proximate a back region of said disposable diaper.

9. A disposable diaper in accordance with claim 8, wherein said flap sheet comprises at least two discrete pleats disposed along opposed sides of said pocket.

10. A disposable diaper in accordance with claim 8, wherein said pocket substantially traverses an entire width of said back region of said disposable diaper.

11. A disposable diaper in accordance with claim 8, wherein said flap sheet comprises a nonwoven web.

12. A disposable diaper in accordance with claim 11, wherein said nonwoven web is selected from the group consisting of spunbond, meltblown, bonded carded web and combinations thereof.

13. A disposable diaper in accordance with claim 8, wherein said fluid permeable body-side liner is a multi-layer material comprising an absorbent layer and a fluid permeable top sheet, said absorbent layer being disposed between said fluid impermeable outer cover and said fluid permeable top sheet.

14. A disposable diaper in accordance with claim 8, wherein said flap sheet is fluid impermeable.

15. A method for producing a disposable absorbent article comprising the steps of:
forming a substantially rectangular multi-layer material comprising a fluid impermeable backsheet, a fluid permeable body-side liner attached to one side of said fluid impermeable backsheet, and a flap sheet attached to said fluid permeable body-side liner whereby said body-side liner is disposed between said fluid impermeable backsheet and said flap sheet;
removing a portion of said material from opposed longitudinal edges of said substantially rectangular multi-layer material, producing two enlarged end portions and a narrowed portion intermediate said two enlarged end portions, one of said enlarged end portions corresponding to a back region of said disposable absorbent article; and
forming at least two discrete longitudinal pleats in said flap sheet proximate said back region.

16. A method in accordance with claim 15, wherein at least one layer of said multi-layer material comprises a nonwoven material.

17. A method in accordance with claim 16, wherein said nonwoven material is selected from the group consisting of spunbond, meltblown, bonded carded web and combinations thereof.
